(19) United States
(12) Patent Application Publication
(10) Pub. No.: US 2012/0330053 A1
(43) Pub. Date: Dec. 27, 2012

(54) METHOD FOR PRODUCING ACYLATE

(75) Inventors: Tomoya HOSODA, Tsukuba-shi, Ibaraki (JP); Eiji HOSODA, Niihama-shi (JP); Masanobu MATSUBARA, Niihama-shi (JP)

(73) Assignee: SUMITOMO CHEMICAL COMPANY, LIMITED, Tokyo (JP)

(21) Appl. No.: 13/489,503

(22) Filed: Jun. 6, 2012

(30) Foreign Application Priority Data
Jun. 27, 2011 (JP) 2011-142151

(51) Int. Cl. C07C 67/08

(52) U.S. Cl. 560/255

(57) ABSTRACT

A method for producing an acylate by acylating a phenolic hydroxyl group contained in an aromatic hydroxycarboxylic acid and/or an aromatic diol with a fatty acid anhydride in an acylation reaction vessel, the method including refluxing a liquid, which is obtained by cooling an evaporant from the acylation reaction vessel, so as to allow the liquid to flow down an inner wall surface of the acylation reaction vessel in an amount of 10 kg/hour/m or more.

### BACKGROUND OF THE INVENTION

1. Field of the Invention

The present invention relates to a method for producing an acylate.

2. Description of the Related Art

Among monomers used in the production of a liquid crystal polyester, a monomer having a phenolic hydroxyl group, such as an aromatic hydroxycarboxylic acid or an aromatic diol, has low reactivity. It has been known that the monomer is acylated with a fatty acid anhydride to obtain an acrylate and the acrylate is used as a monomer so as to enhance reactivity of the phenolic hydroxyl group (see, for example, JP-A-2002-146003 and its corresponding application US2002/0055607A).

### SUMMARY OF THE INVENTION

However, the above method for producing an acrylate has problems that (1) since raw compounds cannot undergo an acylation reaction in a desired amount ratio, a liquid crystal polyester having excellent physical properties (for example, heat resistance) cannot be obtained, and (2) a liquid crystal polyester cannot be obtained in satisfactory yield. An object of the present invention is to provide a method for producing an acrylate in which the problems (1) and (2) have been solved.

The present invention relates to a method for producing an acylate by acylating a phenolic hydroxyl group contained in an aromatic hydroxycarboxylic acid and/or an aromatic diol with a fatty acid anhydride in an acylation reaction vessel, the method including refluxing a liquid, which is obtained by cooling an evaporant from the acylation reaction vessel, so as to allow the liquid to flow down on an inner wall surface of the acylation reaction vessel in an amount of 10 kg/hour/m or more.

In a preferred aspect of the present invention, the aromatic hydroxycarboxylic acid and the aromatic diol are respectively compounds represented by the following formulas (1′) and (2′):

HO—Ar¹—COOH (1′)

HO—Ar³—OH (2′)

wherein Ar¹ is a 2,6-naphthylene group, a 1,4-phenylene group, or a 4,4′-biphenylylene group; Ar³ is a 2,6-naphthylene group, a 1,4-phenylene group, a 1,3-phenylene group, or a 4,4′-biphenylylene group; and one or more hydrogen atoms among Ar¹ and Ar³, each independently may be substituted with a halogen atom, an alkyl group, or an aryl group.

### BRIEF DESCRIPTION OF THE DRAWINGS

FIG. 1 is a schematic view showing an example of an apparatus which can be used in the present invention. The size and proportion of each component are not necessarily the same as actual size and proportion for simplicity of the drawing.

### DETAILED DESCRIPTION OF THE INVENTION

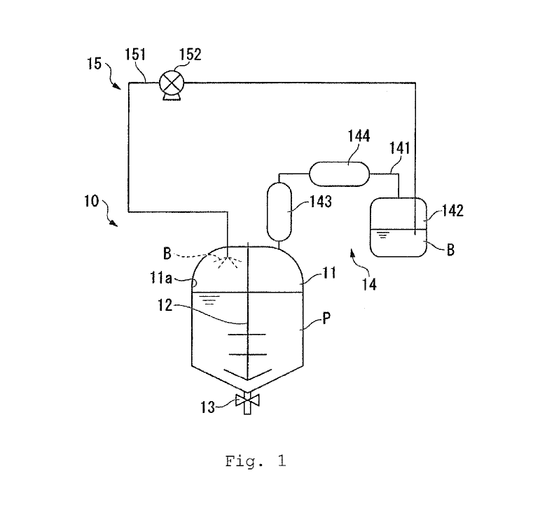

An apparatus 10 shown in FIG. 1 includes an acylation reaction vessel 11 which also serves as a melt polycondensation vessel, a stirrer 12, and a valve 13 for controlling a discharge amount of polycondensate. Hereinafter, “melt polycondensation” is simply referred to as “polymerization”.

A recovery device 14, which recovers an evaporant containing by-products formed in step (1) by cooling, is provided at the upper portion of the acylation reaction vessel 11. The recovery device 14 includes a piping 141 whose one end is connected to the acylation reaction vessel 11, and whose other end is connected to a tank 142. In the piping 141, a first cooler 143 and a second cooler 144, which cool an evaporant from the acylation reaction vessel 11, are provided.

The recovery device 14 is connected to a spraying device 15. The spraying device 15 includes a piping 151 whose one end is connected to the tank 142, and whose other end is connected to the acylation reaction vessel 11. In the piping 151, a pump 152, which transfers a liquid accumulated in the tank 142, is provided. A part of the liquid accumulated in the tank 142 is sprayed over an inner wall surface 11a of the acylation reaction vessel 11 by the spraying device 15.

Examples of the liquid crystal polyester, which can be produced using an acrylate produced by the method of the present invention, include the following liquid crystal polyesters:
(I) a liquid crystal polyester which is obtained by polymerizing an acrylate of an aromatic hydroxycarboxylic acid and/or an acylate of an aromatic diol with an aromatic dicarboxylic acid or a polymerizable derivative thereof; and
(II) a liquid crystal polyester which is obtained by polymerizing an acrylate of an aromatic hydroxycarboxylic acid and/or an acylate of an aromatic diol with a polyester such as polyethylene terephthalate.

Examples of the polymerizable derivative of the aromatic dicarboxylic acid include a derivative (ester) in which a carboxyl group of an aromatic dicarboxylic acid is converted into an alkoxycarbonyl group or an aryloxycarbonyl group, a derivative (acid halide) in which a carboxyl group is converted into a haloformyl group, and a derivative (acid anhydride) in which a carboxyl group is converted into an acyloxycarbonyl group.

There is no particular limitation on the fatty acid anhydride according to the present invention. Examples of the fatty acid anhydride include acetic anhydride, propionic anhydride, butyric anhydride, isobutyric anhydride, valeric anhydride, pivalic anhydride, 2-ethylhexanoic anhydride, monochloroacetic anhydride, dichloroacetic anhydride, trichloroacetic anhydride, monobromoacetic anhydride, dibromoacetic anhydride, tribromoacetic anhydride, monofluoroacetic anhydride, difluoroacetic anhydride, trifluoroacetic anhydride, glutaric anhydride, maleic anhydride, succinic anhydride and β-bromopropionic anhydride; and two or more combinations thereof. Among these fatty acid anhydrides, acetic anhydride, propionic anhydride, butyric anhydride or isobutyric anhydride is preferable and acetic anhydride is more preferable from the viewpoint of costs and handling properties.

The use amount of the fatty acid anhydride is preferably from 1.00 to 1.20 equivalents based on 1.00 equivalent of the phenolic hydroxyl group of the aromatic hydroxycarboxylic acid and/or the aromatic diol. The use amount of the fatty acid anhydride is more preferably from 1.00 to 1.05 equivalents, and still more preferably from 1.03 to 1.05 equivalents, from the viewpoint of less outgassing from a molded article of a liquid crystal polyester and performances such as solder blister resistance of a molded article. From the viewpoint of impact strength of a molded article, the use amount is more preferably from 1.05 to 1.10 equivalents.

When the use amount of the fatty acid anhydride is less than 1.00 equivalent, equilibrium of the acylation reaction shifts to the fatty acid anhydride side. As a result, sublimation of the aromatic diol and/or aromatic dicarboxylic acid, which are/is not acylated, may cause clogging of the acylation reaction vessel. When the use amount of the fatty acid anhydride is more than 1.20 equivalents, the liquid crystal polyester to be produced by using the obtained acrylate may cause severe coloration.

The acylation reaction is preferably performed under the conditions at 130 to 180° C. for 30 minutes to 20 hours, and more preferably 140 to 160° C. for 1 to 5 hours.

The acylation reaction vessel is a vessel which also serves as a polymerization vessel for the production of a liquid crystal polyester, or a vessel which is different from a polymerization vessel. The former aspect is a preferable aspect from the viewpoint of (i) no need of the polymerization vessel, and (ii) simple operation since the acylation and polymerization are performed using the same vessel.

Examples of the material of the acylation reaction vessel include materials having corrosion resistance, such as titanium and hastelloy B. In the case of producing a liquid crystal polyester having high color tone (L value), the material of the inner wall of the reaction vessel is preferably glass. Examples of the reaction vessel in which the material of the inner wall is glass include a reaction vessel which is entirely made of glass, a reaction vessel in which only an inner wall of the portion in contact with the reaction mixture is made of a glass, and a reaction vessel made of SUS whose inner wall is glass-lined. Among these reaction vessels, a reaction vessel, made of SUS, whose inner wall is glass-lined is preferable in a large-sized production facility. The inner wall of the vessel shown in FIG. 1, which serves both the acylation reaction vessel and the polymerization vessel 11, is glass-lined.

Since aromatic compounds such as an aromatic hydroxycarboxylic acid and an aromatic diol are likely to be solidified, they are likely to adhere on an inner wall surface of the acylation reaction vessel. The adhesion may cause problems such as (1) deterioration of physical properties such as heat resistance of the obtained liquid crystal polyester since raw compounds cannot be subjected to an acylation reaction in a desired amount ratio, and (2) a decrease in yield of the liquid crystal polyester.

In the present invention, the above-mentioned problems (1) and (2) are solved by controlling a reflux amount of a liquid which flows down on an inner wall surface of the acylation reaction vessel to 10 kg/hour/m or more. The liquid, which flows down on the inner wall surface, contains an acylate, an unreacted aromatic hydroxycarboxylic acid, an unreacted aromatic diol, an unreacted fatty acid anhydride and a by-produced fatty acid. The reflux amount (kg) means the amount per 1 m of the length in a horizontal direction on the inner wall surface 11a of the acylation reaction vessel 11, and per 1 hour.

There is no particular limitation on the method in which a liquid obtained by cooling an evaporant from an acylation reaction vessel is allowed to flow down so that the reflux amount becomes 10 kg/hour/m or more. Examples of the method include a method using a spraying device 15 shown in FIG. 1. The reflux amount by this method is calculated based on a flow rate per 1 hour of a pump 152, and can be set to a desired value by changing a flow rate of the pump. In the case where the flow rate of the liquid, which is cooled by the first cooler 143 or the second cooler 144 and then directly refluxed into the acylation reaction vessel, is negligibly small, the reflux amount is calculated based only on the low rate of the pump 152. In the present invention, the production method is preferably a method in which flow-down of a liquid obtained by cooling an evaporant is performed by a spraying device for spraying the liquid over the inner wall surface of the acylation reaction vessel.

Another method of adjusting the reflux amount to 10 kg/hour/m or more includes a method in which a control is made so that the liquid formed in the first cooler 143 or the second cooler 144 directly flows down on the inner wall surface of the acylation reaction vessel in a reflux amount of 10 kg/hour/m or more without passing through the spraying device 15. The reflux amount can be determined by the following procedure. That is, the state of the flow-down is photographed through an inspection hole provided in the acylation reaction vessel and the obtained image is analyzed, and thus a flow rate per 1 m of a perimeter of the inner wall surface per 1 hour can be calculated. The above-mentioned two flow-down methods may be mutually combined so that the reflux amount is 10 kg/hour/m or more. In this case, the reflux amount is the total of reflux amounts in the respective methods and is controlled so that the total is 10 kg/hour/m or more.

When the reflux amount is less than 10 kg/hour/m, it is impossible to sufficiently decrease the amount of an aromatic compound adhered on the inner wall surface of the acylation reaction vessel. The reflux amount is preferably from 10 to 100 kg/hour/m, and more preferably from 10 to 50 kg/hour/m.

Reflux of the liquid accumulated in the tank 142 to the acylation reaction vessel by the spraying device 15 is performed during or after the acylation reaction.

The production of a liquid crystal polyester using an acrylate is performed by polycondensation (melt polycondensation) of an acrylate with a compound represented by the following formula (3′) under heating and stirring in a molten state:

G²—CO—Ar²—CO—G² (3′)

wherein Ar² is a 2,6-naphthylene group, a 1,4-phenylene group, a 1,3-phenylene group or a 4,4′-biphenylylene group; G² each independently represents a hydroxyl group, an alkoxy group, an aryloxy group, an alkylcarbonyloxy group or a halogen atom; and one or more hydrogen atoms in Ar² each independently may be substituted with a halogen atom, an alkyl group or an aryl group.

Symbols shown in the formulas (1′), (2′) and (3′) will be described below.

Examples of the halogen atom include a fluorine atom, a chlorine atom, bromine atom and an iodine atom.

Examples of the alkyl group include a methyl group, an ethyl group, a n-propyl group, an isopropyl group, a n-butyl group, a isobutyl group, a sec-butyl group, a tert-butyl group, a n-pentyl group, a n-hexyl group, a n-heptyl group, a 2-ethylhexyl group, a n-octyl group, a n-nonyl group and a n-decyl group. The number of carbon atoms is preferably from 1 to 10.

Examples of the aryl group include a phenyl group, an o-tolyl group, a m-tolyl group, a p-tolyl group, a 1-naphthyl group and a 2-naphthyl group. The number of carbon atoms is preferably from 6 to 20.

In the case where hydrogen atoms in Ar¹, Ar² or Ar³ are substituted with the above group, the number of substituents, each independently, is preferably 2 or less, and more preferably 1.

Examples of the alkoxy group of G² include monovalent groups in which the above-mentioned alkyl group and an oxygen atom (—O—) are combined, such as a methoxy group and an ethoxy group.

Examples of the aryloxy group of G² include monovalent groups in which the above-mentioned aryl group and an oxygen atom (—O—) are combined, such as a phenoxy group.

Examples of the alkylcarbonyloxy group of G² include monovalent groups in which the above-mentioned alkyl group and a carbon atom of a carbonyloxy group (—C(|O)—O—) are combined, such as a methylcarbonyloxy group and an ethylcarbonyloxy group.

Examples of the halogen atom of G² include a chlorine atom, a bromine atom and an iodine atom.

While the use amount of each monomer in the polymerization will be described below, the compounds represented by the formulas (1′), (2′) and (3′) are respectively referred to as a monomer (1′), a monomer (2′) and a monomer (3′). The monomer (1′) and the monomer (2′) are used as an acrylate thereof in the polymerization, as a matter of course.

The use amount of the monomer (1′) is preferably 30 mol % or more, more preferably from 30 to 80 mol %, still more preferably from 40 to 70 mol %, and particularly preferably from 45 to 60 mol %, based on 100 mol % in total of the use amounts of the monomers (1′), (2′) and (3′).

Each use amount of the monomers (2′) and (3′) is preferably 35 mol % or less, more preferably from 10 to 35 mol %, still more preferably from 15 to 30 mol %, and particularly preferably from 17.5 to 27.5 mol %, based on 100 mol % in total of the use amounts of the monomers (1′), (2′) and (3′).

When the use amount of the monomer (1′) is 30 mol % or more, heat resistance, strength and rigidity of the obtained liquid crystal polyester are likely to be improved. However, when the use amount is more than 80 mol %, solubility of the obtained liquid crystal polyester in a solvent is likely to decrease.

The use amount of the monomer in which Ar¹, Ar² or Ar³ is a 2,6-naphthylene group is preferably 10 mol % or more, and more preferably 40 mol % or more, based on 100 mol % in total of the use amounts of the monomers (1′), (2′) and (3′).

The ratio of the use amount (mol) of the monomer (2′) to that of the monomer (3′), namely, [use amount of the monomer (2′)/[use amount of the monomer (3′)] is preferably from 0.9/1 to 1/0.9, more preferably from 0.95/1 to 1/0.95, and still more preferably from 0.98/1 to 1/0.98.

The monomers (1′) to (3′) each independently may be a combination of two or more compounds. The monomer other than the monomers (1′) to (3′) may also be used, and the use amount the other monomer is preferably 10 mol % or less, and more preferably 5 mol % or less, based on 100 mol % in total of the use amounts of all monomers.

The polymerization may be carried out in the presence of a catalyst, and examples of the catalyst include metal compounds such as magnesium acetate, stannous acetate, tetrabutyl titanate, lead acetate, sodium acetate, potassium acetate and antimony trioxide; and nitrogen-containing heterocyclic compounds such as 4-(dimethylamino)pyridine and 1-methylimidazol. Among these catalysts, nitrogen-containing heterocyclic compounds are preferably used. The use of the catalyst, and the kind of the catalyst when used may be determined depending on applications of a liquid crystal polyester. For example, a liquid crystal polyester to be used for food applications is preferably produced in the absence of the catalyst. In the case of the liquid crystal polyester produced by using the catalyst, a catalyst component contained therein must be sometimes removed depending on applications.

The polymerization can be performed in an atmosphere of an inert gas such as nitrogen under the condition of a normal or reduced pressure. It is particularly preferred that the polymerization is performed in an inert gas atmosphere under a normal pressure. The polymerization is performed in a batch-wise or continuous manner or a combination thereof.

The polymerization temperature is usually from 260 to 350° C., and preferably from 270 to 330° C. When the polymerization temperature is lower than 260° C., the polymerization proceeds slowly. In contrast, when the polymerization temperature is higher than 350° C., side reactions such as decomposition of the polymer are likely to occur. When the polymerization vessel is composed of a plurality of divisions divided into multi-stages or partitioned plural divisions and the polymerization temperature of each division is not the same, the highest temperature among them means the polymerization temperature.

While the polymerization time should be appropriately determined based on other reaction conditions, the polymerization time is preferably from 0.5 to 5 hours at the above polymerization temperature.

The polymerization vessel may be a polymerization vessel having a known shape. In the case of a vertical polymerization vessel, a stirring blade is preferably a multi-stage paddle blade, a turbine blade, a monte blade or a double helical blade, and more preferably a multi-stage paddle blade or a turbine blade. A lateral polymerization vessel is preferably a polymerization vessel provided with a blade having a specific shape, such as a lens blade, an eyeglass blade or an elliptical flat-plate blade in a vertical direction of a single or twin stirring shaft. In order to improve stirring performances and feed mechanism, the blade may be provided with torsion.

The polymerization vessel is heated by a heat medium, a gas or an electric heater. In order to uniformly heat a reaction mixture in the polymerization vessel, not only the polymerization vessel, but also members to be immersed in the reaction mixture, such as a stirring shaft, a blade and a baffle plate are preferably heated.

The polymer (liquid crystal polyester) obtained by polymerization using an acrylate includes a repeating unit derived from the monomer (1′) represented by the following formula (1) (hereinafter referred to as a “repeating unit (1)”) and/or a repeating unit derived from the monomer (2′) represented by the following formula (2) (hereinafter referred to as a “repeating unit (2)”), and a repeating unit derived from the monomer (3′) represented by the following formula (3) (hereinafter referred to as a “repeating unit (3)”), and preferably includes a repeating unit (1):

—O—-Ar¹—CO— (1)

—O—Ar³—O— (2)

—CO—Ar²—CO—. (3)

The repeating unit (1) is a repeating unit derived from an aromatic hydroxycarboxylic acid. The repeating unit (1) is preferably a repeating unit derived from p-hydroxybenzoic acid in which Ar¹ is a p-phenylene group, or a repeating unit derived from 6-hydroxy-2-naphthoic acid in which Ar¹ is a 2,6-naphthylene group.

The repeating unit (2) is a repeating unit derived from an aromatic diol. The repeating unit (2) is preferably a repeating unit derived from hydroquinone in which Ar³ is a p-phenylene group, or a repeating unit derived from 4,4′-dihydroxybiphenyl in which Ar³ is a 4,4′-biphenylylene group.

The repeating unit (3) is a repeating unit derived from an aromatic dicarboxylic acid. The repeating unit (3) is preferably a repeating unit derived from terephthalic acid in which Ar² is a p-phenylene group, a repeating unit derived from isophthalic acid in which Ar² is a m-phenylene group, a repeating unit derived from 2,6-naphthalenedicarboxylic acid in which Ar² is a 2,6-naphthylene group, or a repeating unit derived from diphenylether-4,4′-dicarboxylic acid in which Ar² is a diphenylether-4,4′-diyl group.

In the liquid crystal polyester, the total amount of repeating units having a 2,6-naphthylene group is preferably 10 units or more, and more preferably 40 units or more, based on 100 units of the total amount of all repeating units.

Examples of the liquid crystal polyester having high heat resistance and melt tension include liquid crystal polyesters which satisfy at least one of the following conditions (I) to (V) (unless otherwise specified, the total amount of the repeating units (1), (2) and (3) is 100 units):
(I) the content of a repeating unit (1) in which Ar¹ is a 2,6-naphthylene group is preferably from 40 to 74.8 units, more preferably from 40 to 64.5 units, and still more preferably from 50 to 58 units;
(II) the content of a repeating unit (2) in which Ar³ is a 1,4-phenylene group is preferably from 12.5 to 30 units, more preferably from 17.5 to 30 units, and still more preferably from 20 to 25 units;
(III) the content of a repeating unit (3) in which Ar² is a 2,6-naphthylene group (hereinafter referred to as a “repeating unit (3A)”) is preferably from 12.5 to 30 units, more preferably from 17.5 to 30 units, and still more preferably from 20 to 25 units;
(IV) the content of a repeating unit (3) in which Ar² is a 1,4-phenylene group (hereinafter referred to as a “repeating unit (3B)”) is preferably from 0.2 to 15 units, more preferably from 0.5 to 12 units, and still more preferably from 2 to 10 units; and
(V) the amount of the repeating unit (3A) is preferably 0 units or more, and more preferably 60 units or more, based on 100 units of the total amount of the repeating unit (3A) and the repeating unit (3B).

Since a polymer (liquid crystal polyester) obtained by polymerization using an acrylate is usually solidified by cooling, the molecular weight of the polymer can be increased by crushing the solid matter using a known crushing device and subjecting the obtained powder to solid-phase polymerization under heating of an inert gas atmosphere.

With respect to what degree is solid-phase polymerization carried out, there is a correlation between the molecular weight (polymerization degree) and the flow initiation temperature of a liquid crystal polyester. Therefore, it is possible to exemplify an aspect in which the flow initiation temperature of a liquid crystal polyester to be subjected to solid-phase polymerization is measured and the polymerization degree is ascertained from the measured value, and then the solid-phase polymerization is performed until reaching the flow initiation temperature corresponding to the desired polymerization degree.

The flow initiation temperature is also called a flow temperature and means a temperature at which a viscosity becomes 4,800 Pa·s (48,000 poise) when a liquid crystal polyester is melted while heating at a heating rate of 4° C./min under a load of 9.8 MPa (100 kg/cm²) and extruded through a nozzle having an inner diameter of 1 mm and a length of 10 mm using a capillary rheometer, and the flow initiation temperature serves as an index indicating a molecular weight of the polymer (see “Liquid Crystal Polymer Synthesis, Molding, and Application” edited by Naoyuki Koide, page 95, published by CMC on Jun. 5, 1987).

The liquid crystal polyester obtained by solid-phase polymerization can be preferably granulated into the form of pellets after melting.

Examples of the method of granulating into pellets include a method in which a liquid crystal polyester is melt-kneaded using a commonly used single- or twin-screw extruder, air-cooled or water cooled and then formed into pellets using a pelletizer (strand cutter). Among commonly used extruders, an extruder with large L/D is preferable so as to form after uniformly melting the liquid crystal polyester. The setting temperature (die head temperature) of a cylinder of the extruder is preferably from 200 to 420° C., more preferably from 230 to 400° C., and still more preferably from 240 to 380° C.

Inorganic fillers can be optionally added to the liquid crystal polyester produced by solid-phase polymerization. Examples of the inorganic fillers include calcium carbonate, talc, clay, silica, magnesium carbonate, barium sulfate, titanium oxide, alumina, montmorillonite, gypsum, glass flake, glass fiber, carbon fiber, alumina fiber, silica alumina fiber, aluminum borate whisker and potassium titanate fiber. These inorganic fillers can be used as long as transparency and mechanical strength of the molded body such as a film made of the liquid crystal polyester are not drastically impaired.

It is also possible to optionally add various additives such as an organic filler, an antioxidant, a heat stabilizer, a light stabilizer, a flame retardant, a lubricant, an antistatic agent, an inorganic or organic colorant, a rust preventing agent, a cross-linking agent, a blowing agent, a fluorescent agent, a surface smoothing agent, a surface gloss improver and a mold release improver (for example, fluororesin) to the liquid crystal polyester produced by solid-phase polymerization during the production process of the liquid crystal polyester or processing process after the production.

When an acrylate obtained by the production method of the present invention is used, a liquid crystal polyester can be produced in satisfactory yield without causing deterioration of physical properties such as heat resistance.

One end of the piping 151 shown in FIG. 1 may be connected to the positions before the tank 142, such as the piping 141, the first cooler 143 and the second cooler 144.

### EXAMPLES

While the present invention is descried by way of Examples, the present invention is not limited to these Examples.

### Example 1

A reaction vessel having a capacity of 200 L and an inner diameter of 600 mm, equipped with a stirrer, a nitrogen gas introduction device, a thermometer and a reflux condenser, and a recovery device and a spraying device as shown in FIG. 1 was prepared. A metering pump capable of changing a flow rate, corresponding to the pump 152 shown in FIG. 1, was attached to the spraying device.

In the reaction vessel, 33.1 kg (0.322 kmol) of acetic anhydride was charged under a nitrogen gas atmosphere. Then, 27.9 kg (0.148 kmol) of 2-hydroxy-6-naphthoic acid, 7.4 kg (0.067 kmol) of hydroquinone, 2.2 kg (0.013 kmol) of terephthalic acid, 10.2 kg (0.047 kmol) of 2,6-naphthalenedicarboxylic acid, and 4.8 g of 1-methylimidazole as an acetylation catalyst were charged.

The temperature was raised to 140° C. under a nitrogen gas flow and a flow rate of the metering pump was controlled so that a reflux amount of a liquid, flowing down on an inner wall surface of the reaction vessel, was 16 kg/hour/m, and then an acetylation reaction was carried out at 137 to 140° C. for 1 hour. The reflux amount is the value determined by converting the flow rate of the metering pump into a flow rate per 1 m of a perimeter of an inner wall surface of a reaction vessel per 1 hour.

As a result, the amount of an adhered substance on an inner wall surface of a reaction vessel was about 0.5 kg and this amount was about 0.6% by weight based on 100% by weight of the total charge amount (80.8 kg in total of 33.1 kg of acetic anhydride, 27.9 kg of 2-hydroxy-6-naphthoic acid, 7.4 kg of hydroquinone, 2.2 kg of terephthalic acid and 10.2 kg of 2,6-naphthalenedicarboxylic acid). The amount of the adhered substance (about 0.5 kg) was roughly estimated by the following equation:

Amount of adhered substance=(1) total charge amount −(2) total discharge amount−(3) loss amount

where (1) denotes a total charge amount in a reaction vessel (above-mentioned 80.8 kg); (2) denotes a total discharge amount from the reaction vessel and is 80.3 kg in total; and (3) denotes a loss amount (amount of loss caused by adhesion on a piping or a valve during discharging from the reaction vessel), and 0.01 kg as the value obtained experientially with respect to the device (above-mentioned reaction vessel) was employed.

### Example 2

In the same manner as in Example 1, except that the reflux amount by the metering pump was set to 16 kg/h/m and also acetic acid by-produced during the acetylation reaction was sprayed over the entire surface of the inner wall surface of the reaction vessel, the acetylation reaction was carried out. As a result, an adhered substance was not formed on the inner wall surface of the reaction vessel.

### Comparative Example 1

In the same manner as in Example 1, after raising the temperature to 40° C., the temperature was fallen from 140° C. to 130° C. over 30 minutes in a state where the metering pump was stopped so as not to allow a liquid to flow down on the inner wall surface of the reaction vessel, followed by temperature falling from 130° C. to 125° C. over 30 minutes and further acetylation reaction at 125° C. for 1 hour. As a result, the amount of the adhered substance adhered on the inner wall surface of the reaction vessel was about 3 kg, and was about 3.7% by weight based on 100% by weight of the total charge amount.

The above results revealed that, in Example 1 satisfying “reflux amount<10 kg/hour/m or more” (reflux amount=16 kg/h/m, amount of adhered substance=about 0.5 kg) and Example 2 (reflux amount>16 kg/h/m, amount of adhered substance=0 kg) according to the present invention, the amount of the adhered substance is remarkably decreased as compared with Comparative Example 1 not satisfying the above relations (reflux amount=0 kg/h/m, amount of adhered substance=about 3 kg).

What is claimed is:

1. A method for producing an acylate by acylating a phenolic hydroxyl group contained in an aromatic hydroxycarboxylic acid and/or an aromatic diol with a fatty acid anhydride in an acylation reaction vessel, the method comprising refluxing a liquid, which is obtained by cooling an evaporant from the acylation reaction vessel, so as to allow the liquid to flow down an inner wall surface of the acylation reaction vessel in an amount of 10 kg/hour/m or more.

2. The method according to claim 1, wherein the flow down is carried out by a spraying device for spraying the liquid over the inner wall surface of the acylation reaction vessel.

3. The method according to claim 1, wherein the aromatic hydroxycarboxylic acid and the aromatic diol are respectively compounds represented by the following formulas (1′) and (2′):
HO—Ar²—COOH (2′)
HO—Ar³—OH (2′)
wherein Ar¹ is a 2,6-naphthylene group, a 1,4-phenylene group, or a 4,4′-biphenylylene group; Ar³ is a 2,6-naphthylene group, a 1,4-phenylene group, a 1,3-phenylene group, or a 4,4′-biphenylylene group; and one or more hydrogen atoms among Ar¹ and Ar³, each independently may be substituted with a halogen atom, an alkyl group, or an aryl group.
